(19) United States
(12) Patent Application Publication
(10) Pub. No.: US 2021/0220659 A1
(43) Pub. Date: Jul. 22, 2021

(54) METHOD AND SYSTEM FOR OBTAINING PHYSICAL CONDITION THAT LEAD TO A DEFIBRILLATOR CONUTERSHOCK

(71) Applicants: Nir Geva, Ness Ziona (IL); Ilya Litovchick, Rehovot (IL)

(72) Inventors: Nir Geva, Ness Ziona (IL); Ilya Litovchick, Rehovot (IL)

(73) Assignee: G-Medical Innovations Holdings Ltd, Grand Cayman (KY)

(21) Appl. No.: 16/650,010

(22) PCT Filed: Sep. 24, 2018
(86) PCT No.: PCT/IB18/57354

§ 371 Date: Mar. 24, 2020

Related U.S. Application Data
(60) Provisional application No. 62/562,621, filed on Sep. 25, 2017.

(51) Int. Cl. A61N 1/39

(52) U.S. Cl. CPC A61N 1/3937; A61N 1/36507; A61N 1/3904; A61N 1/3987

(57) ABSTRACT

A method for heart monitoring, the method may include monitoring a heart of a person to provide heart condition information; storing the heart condition information; sensing a provision of a countershock to the person, by a defibrillator, or receiving an indication from the defibrillator about the provision of the countershock; and outputting to an output device that differs from the defibrillator, certain heart condition information related to a certain time period that precedes the provision of the countershock, wherein the certain heart condition information is related to a triggering of the provision of the countershock.

### CROSS REFERENCE

This application claims priority from U.S. provisional patent Ser. No. 62/562621 filing date Sep. 25 2017 which is incorporated herein in its entirety.

### BACKGROUND

Defibrillation (www.wikipedia.org) is a treatment for life-threatening cardiac dysrhythmias, specifically ventricular fibrillation (VF) and non-perfusing ventricular tachycardia (VT).

A defibrillator delivers a dose of electric current (often called a countershock) to the heart. The electrical current does not have to be timed with the heart's intrinsic cardiac cycle. This depolarizes a large amount of the heart muscle, ending the dysrhythmia. Subsequently, the body's natural pacemaker in the sinoatrial node of the heart is able to re-establish normal sinus rhythm.

Various countries require that a defibrillator will be installed in designated premises (such as schools, shopping malls, pools, sport facilities, and the like) that are accessed by members of the public.

The defibrillator monitors the heart rate of the person and based on the heart rate determines whether to generate the countershock. Nevertheless, the condition of the heart—the heart rate pattern—that caused to the defibrillator to generate the countershock is not recorded and the medical staff that treats the person is unaware to condition of the heart prior the countershock and after the countershock.

There is a growing need to provide efficient methods and systems for providing information about the heart condition before and/or after the counter shock.

### SUMMARY

There may be provided a method for heart monitoring the method may include monitoring a heart of a person to provide heart condition information; storing the heart condition information; sensing a provision of a countershock to the person, by a defibrillator, or receiving an indication from the defibrillator about the provision of the countershock; and outputting to an output device that differs from the defibrillator, certain heart condition information related to a certain time period that precedes the provision of the countershock, wherein the certain heart condition information may be related to a triggering of the provision of the countershock.

The monitoring of the heart of the person may be executed by a defibrillator; and wherein the storing of the heart condition information may include storing the heart condition information in a recording device.

The recording device may be integrated with the defibrillator.

The recording device may be mechanically connected to the defibrillator.

The recording device may be detachably coupled to the defibrillator.

The recording device may be mechanically connected to the defibrillator.

The method may include receiving from the defibrillator an indication that the certain heart condition information may be related to the triggering of the provision of the countershock.

The defibrillator may be an automated external defibrillator.

The certain time period precedes a timing of the provision of the countershock by a predetermined time difference.

The monitoring of the heart of the person may include monitoring the heart of the person by a heart monitor that does not belong to the defibrillator, and wherein the heart condition information may be a heart monitor gained heart condition information.

The method may include monitoring the heart rate of the person by the heart monitor, without receiving any information from the defibrillator.

The heart monitor may be detachably connected to the person.

The method may include determining, by the defibrillator and based on defibrillator gained heart condition information, when to provide the countershock.

The heart monitor provided heart condition information may be richer than the defibrillator gained heart condition information.

The method may include determining, by the heart monitor, and based on the heart monitor gained heart condition information, when to provide the countershock.

The method may include instructing the defibrillator, by the heart monitor, when to provide the countershock.

The method may include assigning timestamps to the heart condition information.

The method may include outputting to the output device additional heart condition information related to one or more time periods that differ from the certain time period.

The one or more time periods may include a time period that follows the provision of the countershock.

The method may include outputting an indication that The certain heart condition information may be being stored.

There may be provided a non-transitory computer readable medium that stores instructions that once executed by a computerized system causes the computerized system to perform the steps of monitoring a heart of a person to provide heart condition information; storing the heart condition information; sensing a provision of a countershock to the person, by a defibrillator, or receiving an indication from the defibrillator about the provision of the countershock; and outputting to an output device that differs from the defibrillator, certain heart condition information related to a certain time period that precedes the provision of the countershock, wherein the certain heart condition information may be related to a triggering of the provision of the countershock.

The monitoring of the heart of the person may be executed by a defibrillator; and wherein the storing of the heart condition information may include storing the heart condition information in a recording device.

The non-transitory computer readable medium that stores instructions for receiving from the defibrillator an indication that the certain heart condition information may be related to the triggering of the provision of the countershock.

The defibrillator may be an automated external defibrillator.

The certain time period precedes a timing of the provision of the countershock by a predetermined time difference.

The monitoring of the heart of the person may include monitoring the heart of the person by a heart monitor that does not belong to the defibrillator, and wherein the heart condition information may be a heart monitor gained heart condition information.

The non-transitory computer readable medium that stores instructions for monitoring the heart rate of the person by the heart monitor, without receiving any information from the defibrillator.

The heart monitor may be detachably connected to the person.

The non-transitory computer readable medium that stores instructions for determining, by the defibrillator and based on defibrillator gained heart condition information, when to provide the countershock.

The heart monitor provided heart condition information may be richer than the defibrillator gained heart condition information.

The non-transitory computer readable medium that stores instructions for determining, by the heart monitor, and based on the heart monitor gained heart condition information, when to provide the countershock.

The non-transitory computer readable medium that stores instructions for instructing the defibrillator, by the heart monitor, when to provide the countershock.

The non-transitory computer readable medium that stores instructions for assigning timestamps to the heart condition information.

The non-transitory computer readable medium that stores instructions for outputting to the output device additional heart condition information related to one or more time periods that differ from the certain time period.

The one or more time periods may include a time period that follows the provision of the countershock.

The non-transitory computer readable medium that stores instructions for outputting an indication that The certain heart condition information may be being stored.

There may be provided a system that may include a defibrillator and a recording device, wherein the defibrillator may be configured to (a) monitor a heart of a person to provide heart condition information, (b) determine, based on the heart condition information, whether to generate a countershock, and (c) generate a countershock when determining to generate the countershock; wherein the recording device may be configured to (a) receive from the defibrillator at least certain heart condition information that caused the defibrillator to determine to generate the countershock; (b) store the certain heart condition information; and (c) output the certain heart condition information.

The recording device may be integrated with the defibrillator.

The recording device may be mechanically connected to the defibrillator.

The recording device may be detachably coupled to the defibrillator.

The recording device may be mechanically connected to the defibrillator.

The defibrillator may be configured to send to the recording device an indication that the certain heart condition information may be related to the triggering of the provision of the countershock.

The defibrillator may be an automated external defibrillator.

The certain time period precedes a timing of the provision of the countershock by a predetermined time difference.

The recording device may be configured to output to the output device additional heart condition information related to one or more time periods that differ from the certain time period.

The one or more time periods may include a time period that follows the provision of the countershock.

The recording device may be configured to output an indication that The certain heart condition information may be being stored.

There may be provided a system that may include a defibrillator and a heart monitor that does not belong to the defibrillator; wherein the heart monitor may be configured to monitor a heart of a person to provide heart monitor gained heart condition information; wherein the defibrillator may be configured to monitor the heart of a person to provide defibrillator gained heart condition information; wherein at least one device of the defibrillator and the heart monitor may be configured to determine, based on at least one heart condition information out of the defibrillator gained heart condition information and the heart monitor gained heart condition information, whether to generate a countershock; wherein the defibrillator may be configured to generate a countershock when determining to generate the countershock; and wherein the heart monitor may be configured to output at least certain heart monitor gained heart condition information that caused the defibrillator to determine to generate the countershock.

The heart monitor may be configured to determine, whether to generate a countershock; and to instruct the defibrillator to provide the countershock when determining to provide the countershock.

The defibrillator may be configured to determine, whether to generate a countershock.

The heart monitor may be configured to monitor the heart rate of the person without receiving any information from the defibrillator.

The heart monitor may be detachably connected to the person.

The heart condition information may be richer (be of higher resolution, includes more types of data) than the defibrillator gained heart condition information.

The defibrillator may be an automated external defibrillator.

### BRIEF DESCRIPTION OF THE FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is an example of a person, a defibrillator, a recording device, a network and a server;

FIG. 2 is an example of a person, a defibrillator and a recording device;

FIG. 3 is an example of a person, a defibrillator, a Electrocardiography (ECG) unit, a network and a server;

FIG. 4 is an example of a person, a defibrillator, a ECG unit, a network and a server;

FIG. 5 is an example of a person, a defibrillator, a ECG unit, a network and a server;

FIG. 6 is an example of a person, a defibrillator, a ECG unit, a network and a server;

FIG. 7 is an example of a person, a defibrillator, a ECG unit, one or more other electrodes, a network and a server;

FIG. 8 is an example of a person, a defibrillator, a ECG unit, one or more other electrodes, a network and a server;

FIG. 9 is an example of a person, a defibrillator, a ECG unit, one or more other electrodes, a network and a server;

FIG. 10 is an example of a person, a defibrillator, a ECG unit, one or more other electrodes, a network and a server;

FIG. 11 is an example of a person, a defibrillator, a ECG unit, one or more other electrodes, a network and a server;

FIG. 12 is an example of a person, a defibrillator, a ECG unit, a network and a server;

FIG. 13 is an example of a method; and

FIG. 14 is an example of a person, a defibrillator, a health monitor, a network and a server.

### DETAILED DESCRIPTION OF THE FIGURES

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a system capable of executing the method.

Any reference in the specification to a system should be applied mutatis mutandis to a method that may be executed by the system.

There is provided a system and method for obtaining heart condition information before and/or after an countershock was generated by the defibrillator.

The term “configured to” means “constructed and arranged to”. This may include programming, setting, mechanically amending, and the like.

The terms “health monitor”, “heart monitor” ECG monitor”, and “ECG unit” are used in an interchangeable manner. These terms indicate a unit, device or a system capable of monitoring after, at least, a condition of a heart of a person.

The terms “unit”, “device” and “system” are used in an interchangeable manner.

Heart rate is an example of heart condition information. It may be replaced by any other information regarding a condition of a heart of a person.

The system may or may not include a defibrillator. The system may include a recording device. The system may include a ECG monitor.

Although the following explanation refers to a single countershock the system and method are configured to (applicable to) multiple countershocks.

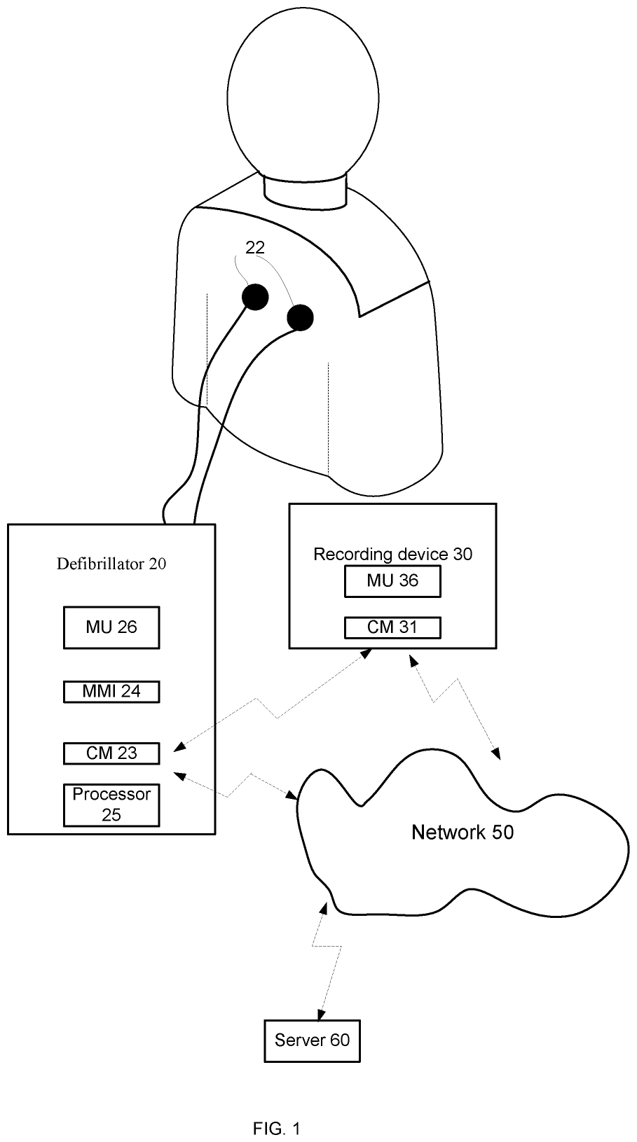

Any communication between any units (defibrillator, recording device, ECG monitor, and the like) can be made over wired and/or wireless channels. Any communication technique may be used—for example short range communication, long range communication, BLUETOOTH™, WI-FI™, cellular communication, satellite communication, and the like. Any cable or wire illustrated in any of the drawings may be replaced by a wireless communication link. The recording device of FIG. 1 and/or any ECG monitor may have one or more plugs such as a USB plug, RSB connectors, or any other communication plug or connectors.

FIG. 1 illustrates an example of person 10, defibrillator 20, recording device 30, network 50 and server 60. The network 50 may include any combination of wired and/or wireless networks. Server 60 is an example of a remote computer. The server 60 may be a cloud computer, may be replaced by multiple computers, and the like.

The defibrillator 20 includes a processor 21, ECG electrodes 22, a communication module (CM) 23, man machine interface (MMI) 24, and memory unit (MU) 26.

The man machine interface 24 may include a screen and/or speaker and/or a microphone for interacting with a person who operates the defibrillator.

The ECG electrodes 22 are mounted on the person 10 and receive electrical detection signals. These detection signals may be processed by processor 21 to determine the heart condition and to determine that a countershock should be provided.

The defibrillator 20 monitors the heart rate of the person 10 via ECG electrodes 22 and sends to the recording device 30 heart condition information that may include at least one out of the detection signals received by the EC electrodes and/or processed information (processed by processor 21).

The defibrillator records countershock information that may include the time of the provision of countershock and may include an intensity of the countershock. The defibrillator may output the countershock information to the recording device and/or to another entity. The countershock information may be stored in memory unit 26.

The heart condition information may include timing information that indicates when the heart condition information was obtained.

The timing information in the heart condition information and in the countershock information enables the medical staff (or any other entity—human or computerized) to determine the heart condition information to the timing of the countershock—and determine the heart condition information before and/or after the countershock.

The defibrillator may send to the recording device the heart condition information and the countershock information.

Alternatively—the heart condition information may include information about the countershock.

The recording device 30 may include (a) a communication unit 31 for communicating with the defibrillator and/or with another entity (such as network 50 and/or server 60) that requests to retrieve the heart condition information stored in the recording device 30, and (b) a memory unit (MU) 36 for storing the heart condition information.

The dashed arrows in FIG. 1 represent communication links. These may be any type of communication links in which any type of communication is used. The communication links may be unidirectional, bidirectional and the like. The arrow directions of FIG. 1 are only an example.

The server 60 may be accessed by the medical staff. Any other device may be coupled to the network 50 or receive the heart condition information.

Electrodes 22 form an ECG lead, and also form a channel for conveying the countershock.

The countershock information may be provided to the recoding device 30.

The recording device 30 may be configured to store but not process the heart rate condition information provided by the defibrillator.

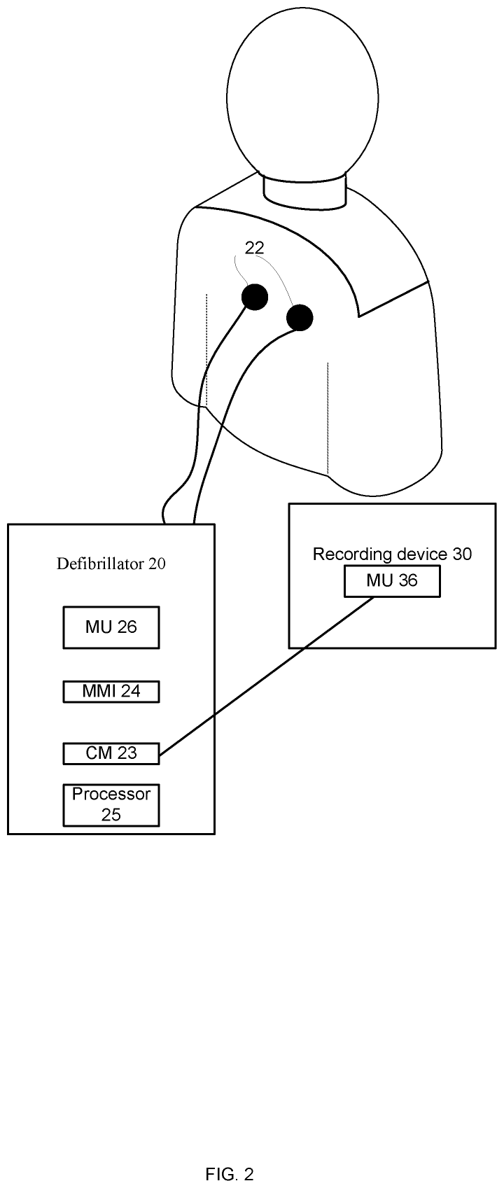

FIG. 2 illustrates an example of person 10, defibrillator 20, recording device 30. The communication units 23 and 31 are linked to each other by a wired communication link.

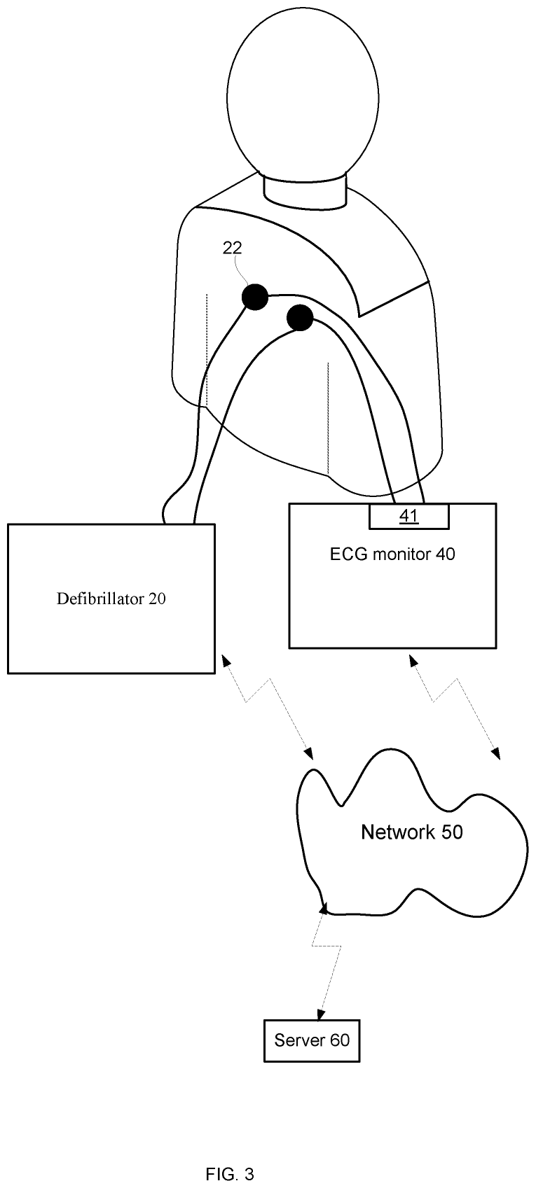

FIG. 3 illustrates an example of person 10, defibrillator 20, ECG monitor 40, network 50 and server 60.

ECG monitor 40 may be electrically coupled (in a wired or wireless manner) to ECG electrodes 22 of the defibrillator—or may otherwise receive the detection signals.

The ECG monitor 40 is configured to receive the detection signals and process them thereby determining the condition of the heart at different points of time. The ECG monitor 40 may receive from defibrillator, heart condition information that differs from the raw signals detected by the electrodes and/or countershock information.

The medical staff (or any other entity) may retrieve the heart condition information from the ECG monitor 40 and the countershock information (from the defibrillator and/or the ECG unit 40)—and determine the heart condition information before and/or after the countershock.

FIG. 3 also illustrates network 50 and server 60. Network 50 may be coupled to ECG monitor 40, defibrillator 20 and server 60. The medical staff may access server 60.

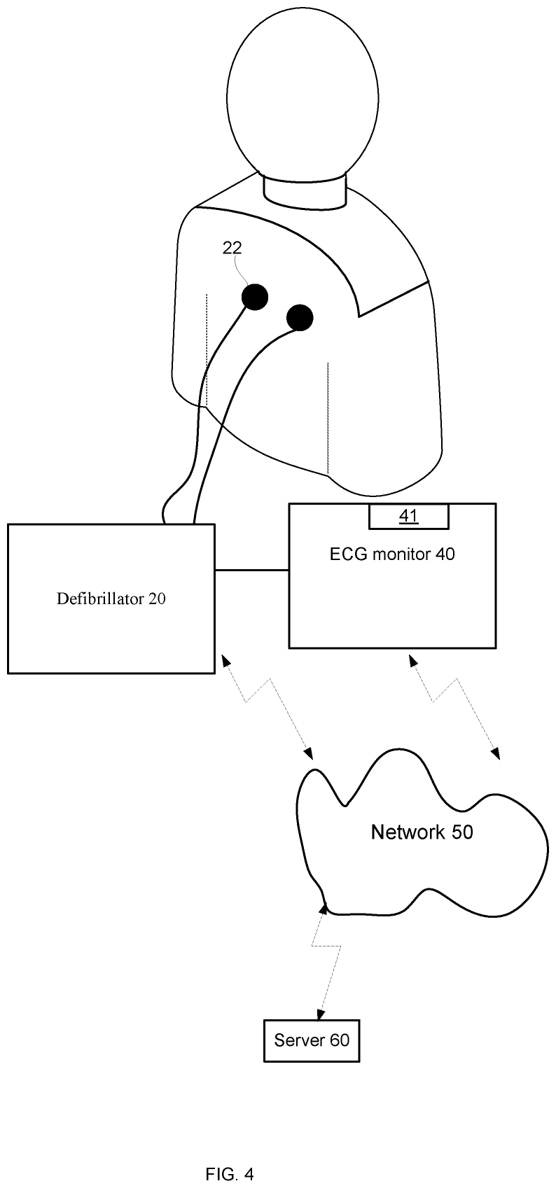

FIG. 4 illustrates an example of person 10, defibrillator 20, ECG monitor 40, network 50 and server 60.

In FIG. 4 the ECG monitor 40 is not coupled to electrodes 22—but receives the raw detection signals sensed by electrodes 22 and/or heart condition information that differs from the raw signals—and may further process this information to determine the heart condition.

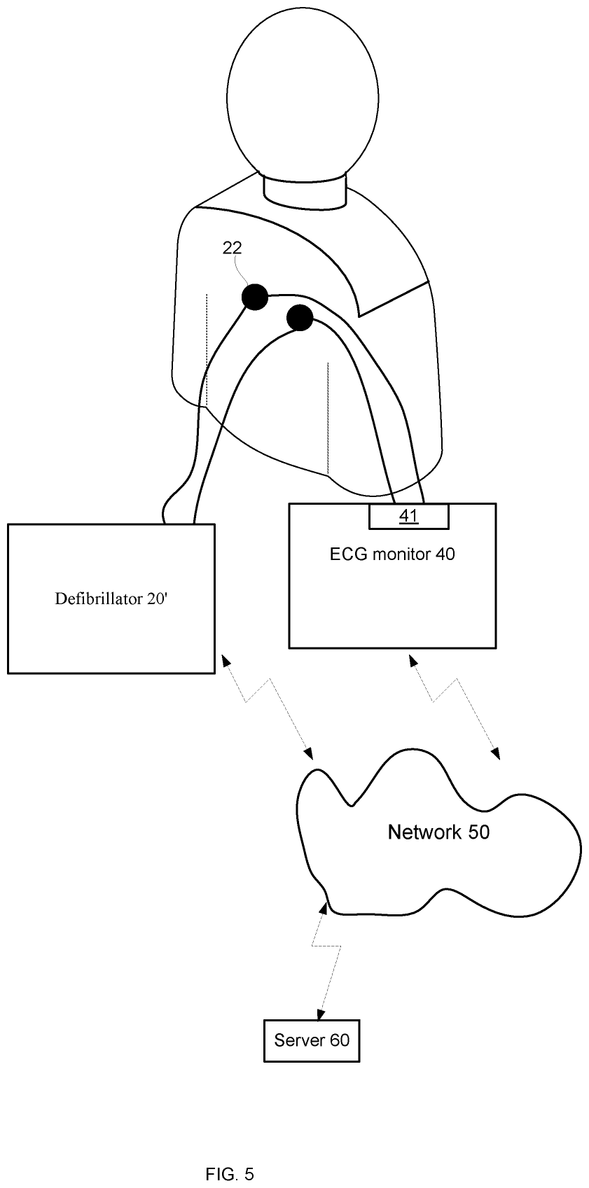

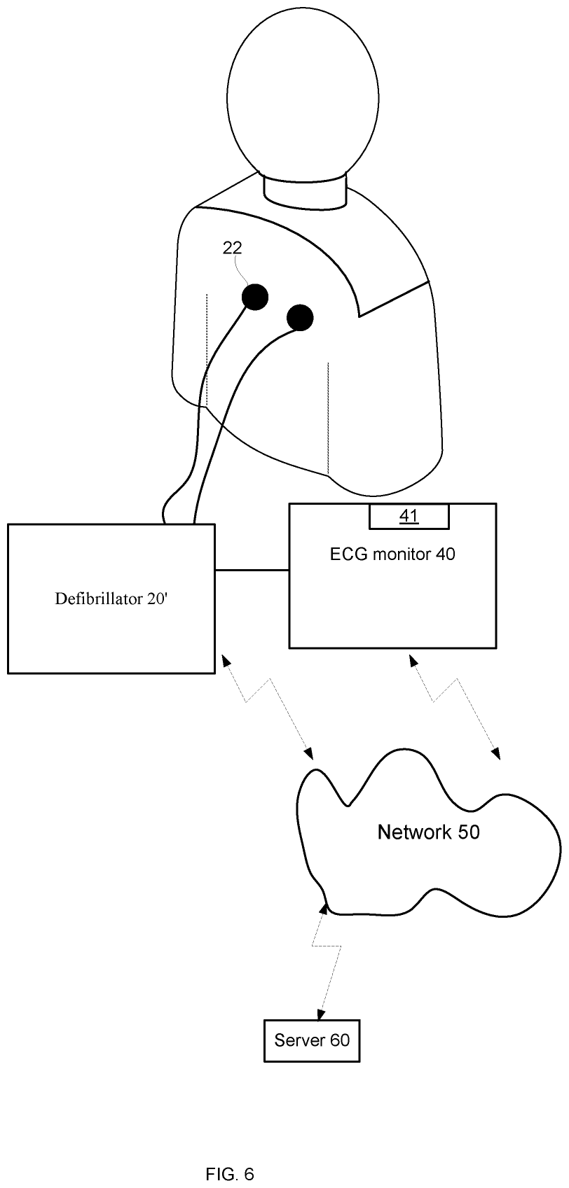

FIGS. 5 and 6 illustrate an example of person 10, defibrillator 20′, ECG monitor 40, network 50 and server 60.

ECG monitor 40 may be electrically coupled (in a wired or wireless manner) to ECG electrodes 22 of the defibrillator (FIG. 5)—or may otherwise receive the detection signals (for example—from the defibrillator—FIG. 6.

Defibrillator 20′ differs from defibrillator 20 of FIG. 20 by lacking (temporarily or constantly) the ability to determine when to generate a counter shock. For example—its processor is not programmed to determine when a countershock should be provided. The processor of the defibrillator 20′ may or may not be programmed to determine the heart condition.

The ECG monitor 40 (or even a remote computer such as server 60) may process the information (at least detection signals from the ECG electrodes 22), determine the heart condition, determine when a countershock should be provided and inform the defibrillator 20′ when the countershock should be provided. The defibrillator may request a person to instruct the defibrillator to generate the countershock and/or may generate the countershock automatically—based on the instructions of the ECG monitor 40.

The ECG monitor 40 may monitor the treatment provided by the defibrillator—especially whether the countershock was generated, what as the effect of the countershock, whether an additional countershock should be generated, and the like.

The ECG monitor 40 may determine the timing, duration and/or strength of the countershock.

The ECG monitor 40 may mark or tag the time of the generation of the countershock.

The ECG monitor 40 may record the countershock (especially when the protecting measures do not disconnect the ECG unit from the electrodes) and may detect the countershock and thus may determine the condition of the heart before and/or after the countershock.

When electrically coupled to electrodes 22, the ECG monitor 40 should be protected against the countershock. The protection measures 41 may include at least one out of a current limiter, a voltage limiter, and a disconnecting element for disconnecting at least one path from the electrodes to the ECG monitor 40. A current limiter may be a circuit with large resistance (for example a resistor having a resistance of few megaohms) that is coupled between the two input ports of the defibrillator that are coupled to the electrodes 22.

The disconnecting element may be a switch or any other circuit that may disconnect the ECG unit (or any internal circuit of the ECG unit) from electrodes 22 based on a sensing (using sensors) of a countershock.

Additionally or alternatively, the ECG unit 40 may receive a notice (for example—from the defibrillator) before the countershock is generated—and temporarily disconnect—at the expected time of the countershock. The disconnection period may be a tradeoff between the need to record the heart condition as close as possible to the countershock—and the need to protect the ECG unit.

Additionally or alternatively, the defibrillator may include protecting measures for disconnecting the ECG unit from the electrodes 22 at the time of the countershock.

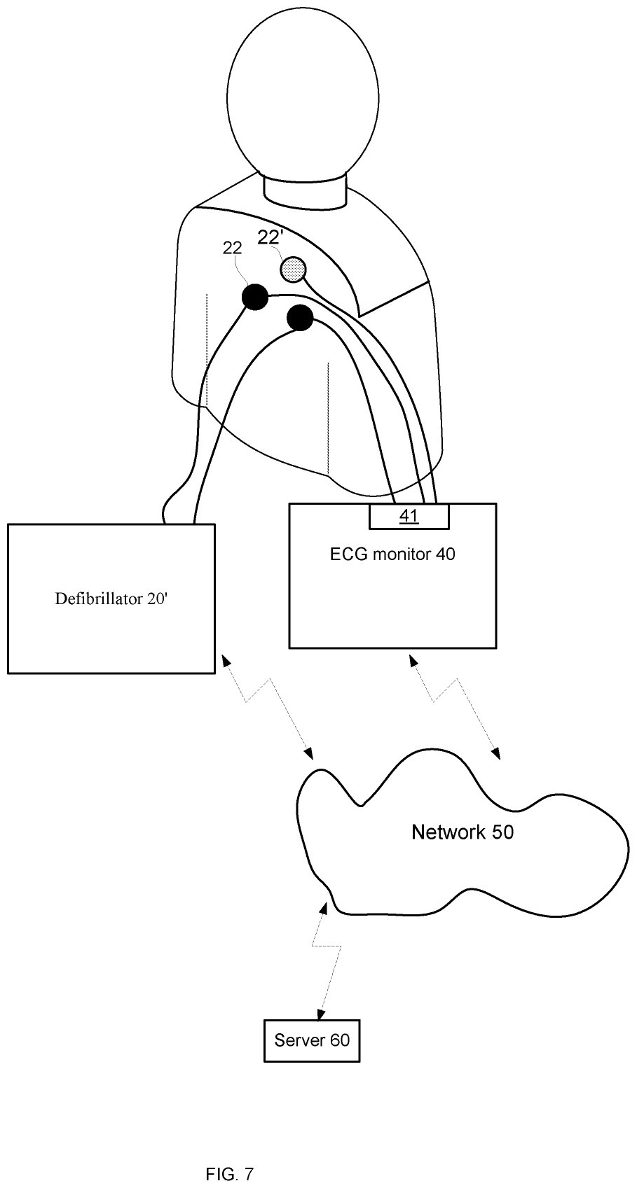

FIG. 7 illustrates an example of person 10, defibrillator 20, ECG monitor 40, other electrode 22′, network 50 and server 60.

Both the defibrillator 20 and the ECG monitor 40 are connected to the person 10.

The ECG monitor 40 may be coupled only to electrodes 22 (of the defibrillator), to one or more electrodes of the defibrillator (as illustrated in FIG. 7) and to at least one more other electrodes—such as other electrode 22′. The number of other electrodes may exceed one.

The connection to one or more other electrodes provide more leads. Lead being the vector between electrodes. The leads may be formed between any combination of electrodes 22 and other electrodes 22′.

The ECG monitor 40 may include protection measures 41—as it should be protected against the countershock.

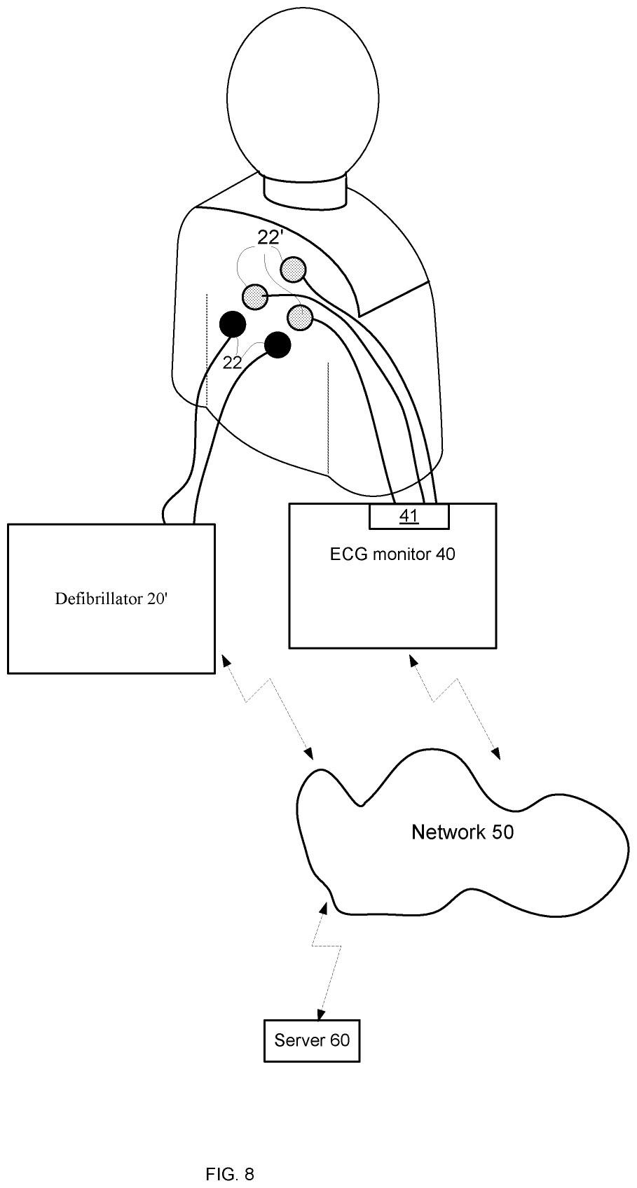

FIG. 8 illustrates an example of person 10, defibrillator 20, ECG monitor 40, other electrodes 22′, network 50 and server 60.

In FIG. 8 the ECG monitor 40 is coupled to other electrodes 22′—and is not electrically coupled to electrodes 22.

The ECG monitor 40 is configured to receive the detection signals and process them thereby determining the condition of the heart at different points of time.

The medical staff (or any other entity) may retrieve the heart condition information from the ECG monitor 40 and the countershock information from the defibrillator—and determine the heart condition information before and/or after the countershock. Alternatively, the information may be retrieved from server 60.

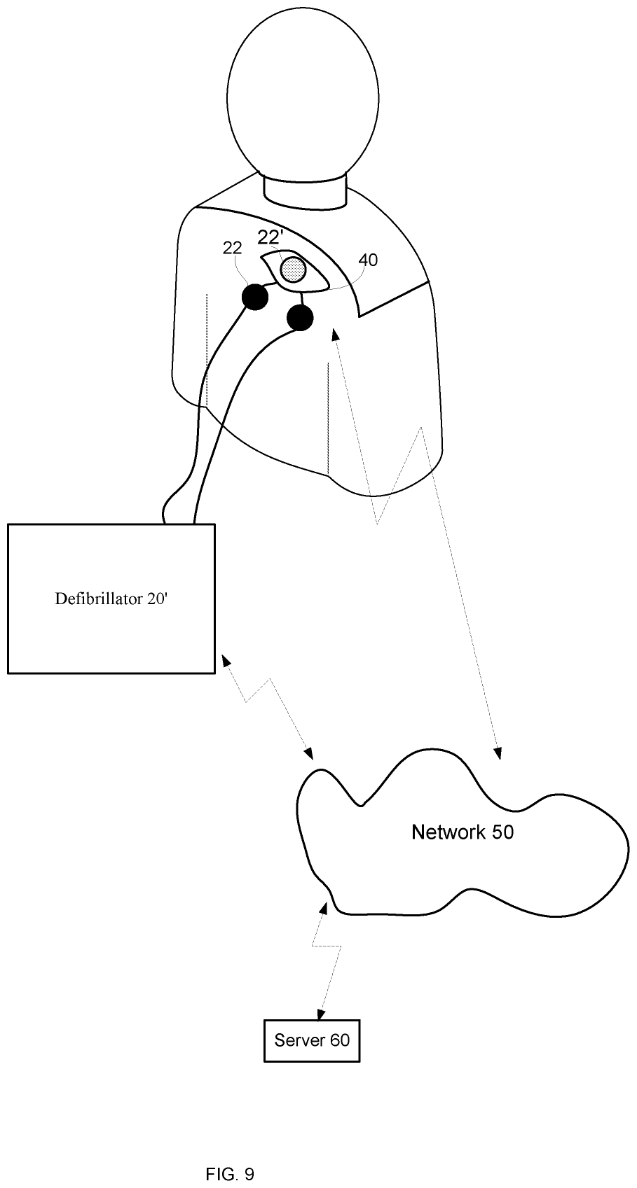

FIG. 9 illustrates an example of person 10, defibrillator 20, ECG monitor 40, other electrode 22′, network 50 and server 60.

In FIG. 9 the ECG monitor 40 is a patch that is coupled to the person. ECG monitor 40 is coupled to electrodes 22 and other electrode 22′.

Other electrode 22′ may be located at the interior surface of the patch—the surface that faces person 10.

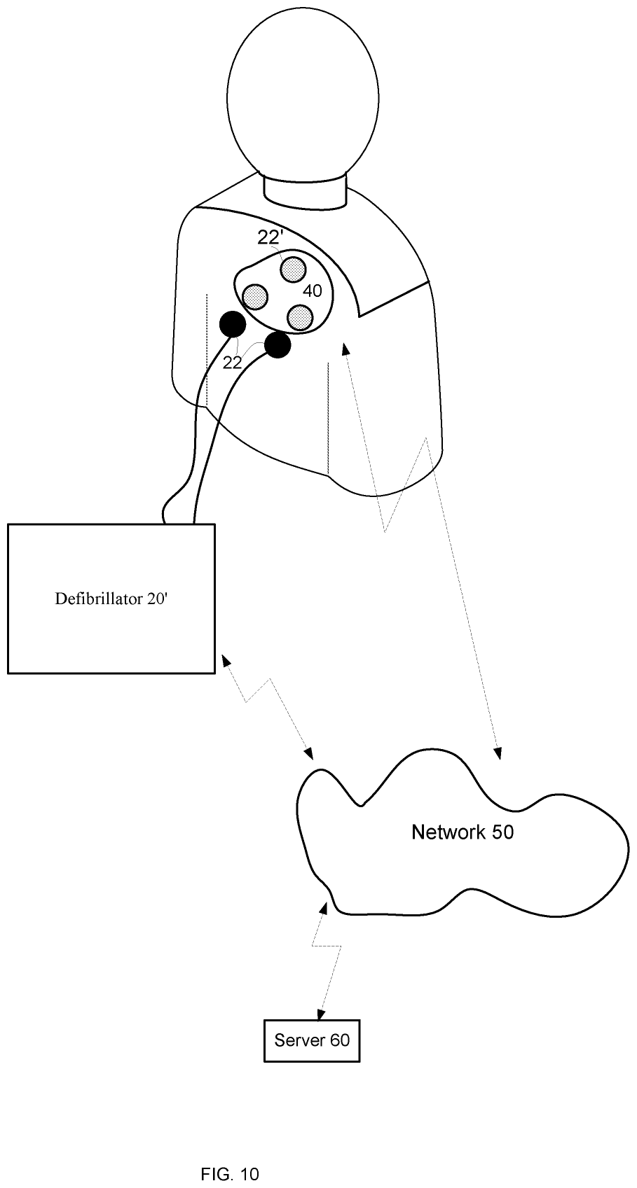

FIG. 10 illustrates an example of person 10, defibrillator 20, ECG monitor 40, other electrodes 22′, network 50 and server 60.

In FIG. 10 the ECG monitor 40 is a patch that is coupled to the person. ECG monitor 40 is coupled to other electrode 22′ and not to electrodes 22.

Other electrodes 22′ may be located at the interior surface of the patch—the surface that faces person 10.

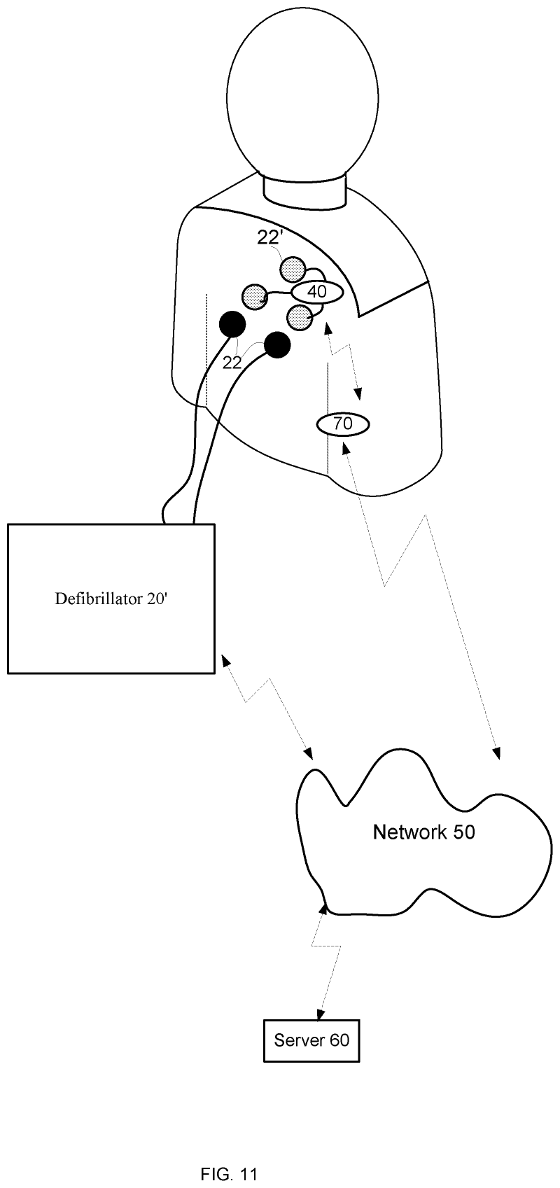

FIG. 11 illustrates an example of person 10, defibrillator 20, ECG monitor 40, other electrodes 22′, network 50 and server 60.

In FIG. 11 the ECG monitor 40 is a patch that is coupled to the person. ECG monitor 40 is coupled to other electrode 22′ and not to electrodes 22.

Other electrodes 22′ are located outside the ECG monitor 40.

FIG. 11 also illustrates an intermediate communication unit 70 that may communicate with ECG monitor 40 and with network. The intermediate communication unit 70 is a non-limiting example of a communication system that may allow the ECG monitor to use short range communication but allow the ECG monitor to communicate with entities that may be located far away. Any of the ECG monitors (and even the recording device 30 of FIG. 10) may communicate with an intermediate communication unit 70.

The intermediate communication unit 70 may be a mobile phone, a relay station, may be included in a mobile phone, and the like.

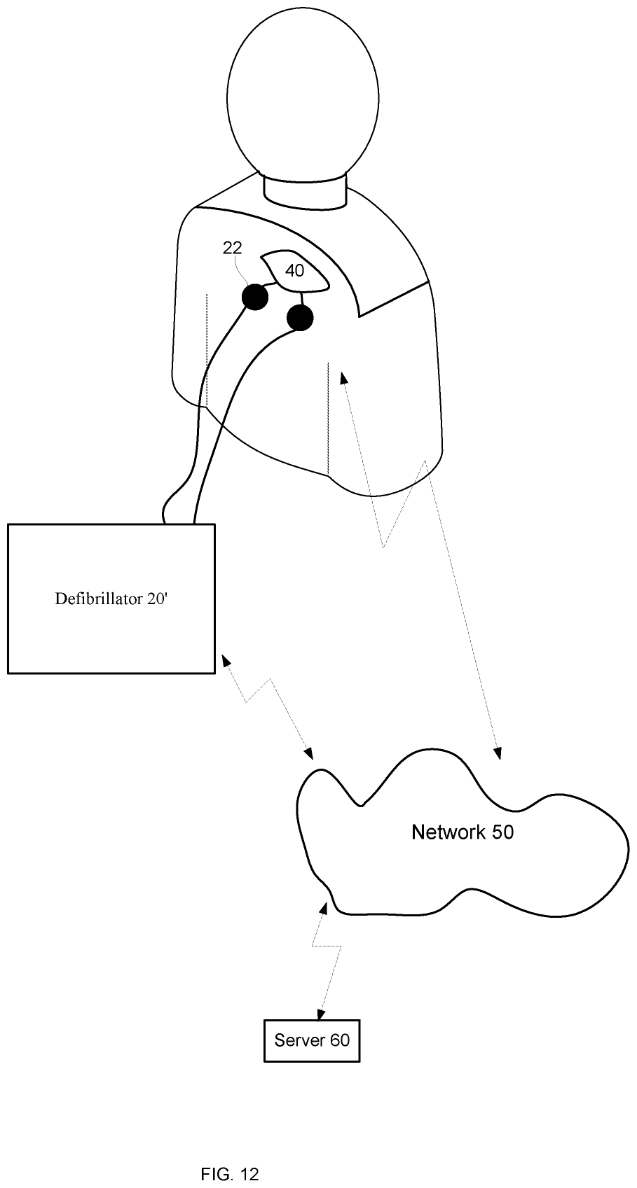

FIG. 12 illustrates an example of person 10, defibrillator 20, ECG monitor 40, network 50 and server 60.

In FIG. 12 the ECG monitor 40 is a patch that is coupled to the person. ECG monitor 40 is coupled to electrodes 22 and not to other electrodes.

Any of the ECG monitors of any of the figures may sense the countershock—as the intensity of the countershock provides an electrical signal that is stronger (even much stronger) than the electrical signals of the heart.

The ECG monitor of any of the figures and/or the recording device 30 may be mechanically connected to the defibrillator, may be detachably coupled to the defibrillator, may be integrated with the defibrillator or may be separated from the defibrillator.

Any defibrillator of FIGS. 1-4 and 7-12 and 14 may be controlled by the ECG monitor 40 or by any remote device. For example, defibrillator 20′ of FIGS. 5 and 6 may replace defibrillator 20.

When the ECG monitor is coupled to one or more other electrodes (electrodes that do not belong to the defibrillator)—then the heart condition information may include more leads and/or be richer and more accurate that the heart condition generated solely by the defibrillator. This (richer) heart condition information may be used to determine when to generate a countershock, and/or determine the parameters of the countershock. This heart condition information may better assist the medical staff.

The heart condition information before and/or after the generation of the countershock may provide the medical staff valuable information that may assist and speed up the medical treatment. For example—knowing the root cause for the heart failure that requited the countershock is of great value.

The heart condition information that is outputted to server 60 (or any other device) may be compressed, may relate to any predefined time period (before and/or after and/or during the provision of the countershock) and/or related to any time period of a predefined timing difference from the timing of the provision of the countershock, and the like. The amount of heart condition information that is being transmitted may be determined based on the transmission conditions and/or available bandwidth and/or criticality of information.

The server and/or any other remote computer may send to the defibrillator and/or ECG monitor and/or recording device requests and/or instructions and/or definitions related to the timing and duration of one or more time windows during which heart condition information is of interest. The timing may indicate the time difference to the provision of the defibrillator. The requests and/or instructions and/or definition may be tailored to certain heart signal patterns (thus may require at least an initial processing of the heart condition information before the transmission to the network)—or may be ignorant to such patterns.

A method may be provided for operating any of the mentioned above devices and/or units. The method may include storing heart condition information before and/or after the countershock, generating heart condition information, detecting a countershock, determining when to generate a countershock and the like.

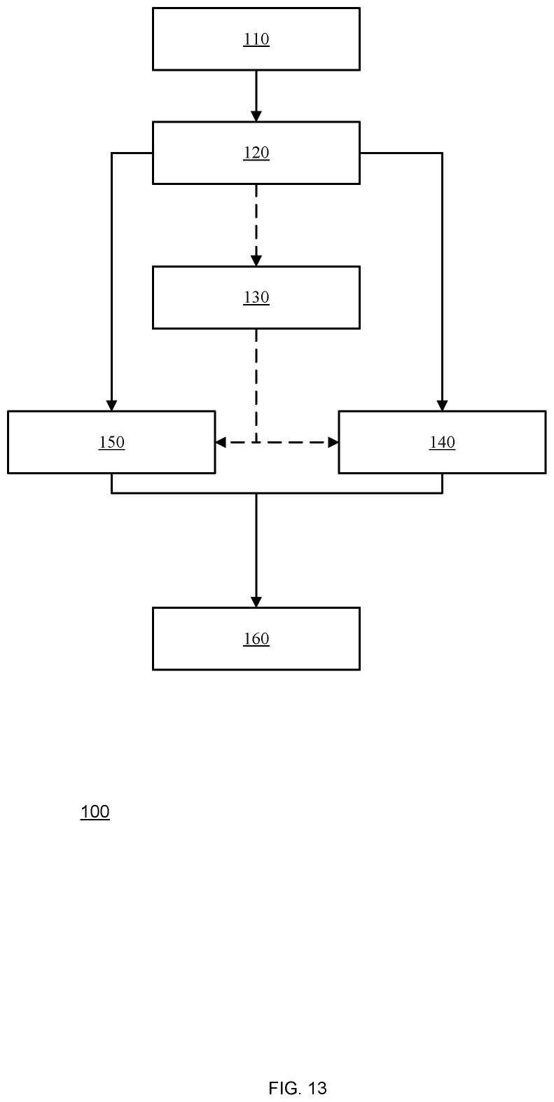

FIG. 13 illustrates method 100 for heart monitoring, the method may include at least some of the listed below steps.

Step 110 of monitoring a heart of a person to provide heart condition information. The monitoring may be executed by the defibrillator to provide defibrillator gained heart condition information. Additionally or alternatively, the monitoring may be executed by a heart monitor to provide heart monitor gained heart condition information.

The heart monitor does not belong to the defibrillator. The heart monitor may be detachably connected to the person.

Step 110 may be executed with or without sharing information and/or signals and/or commands between the defibrillator and the heart monitor.

Step 110 may include assigning timestamps to the heart condition information.

The heart monitor provided heart condition information may be richer than the defibrillator gained heart condition information.

The defibrillator may be an automated external defibrillator. Method 100 may be executed on the field—outside an hospital—without any additional monitoring devices.

Step 110 may be followed by step 120 of storing the storing the heart condition information. The heart condition information may be stored in the heart monitor, in a recording device, and the like.

Step 120 may include storing the heart condition information in a recording device.

The recording device may be integrated with the defibrillator and/or may be mechanically connected to the defibrillator and/or may be detachably coupled to the defibrillator, and/or may be integrated with the defibrillator.

Step 110 may also be followed by at least one of steps 130, 140 and 150.

Step 130 may include determining to provide the countershock and providing the countershock.

Step 130 may include determining, by the defibrillator and based on defibrillator gained heart condition information, when to provide the countershock.

Step 130 may include determining, by the heart monitor, and based on the heart monitor gained heart condition information, when to provide the countershock. This may be followed by instructing the defibrillator, by the heart monitor, when to provide the countershock.

Step 130 may be followed by step 140 and/or 150.

Step 140 may include sensing a provision of a countershock to the person, by the defibrillator. Step 140 may be executed by the defibrillator and/or by the heart monitor.

Step 150 may include receiving an indication from the defibrillator about the provision of the countershock.

Steps 140 and 150 may be followed by step 160 of outputting to an output device that differs from the defibrillator, certain heart condition information related to a certain time period that precedes the provision of the countershock. The certain heart condition information is related to a triggering of the provision of the countershock.

The certain time period may precede a timing of the provision of the countershock by a predetermined time difference (for example—between 1 and 30-seconds). The predetermined time period may be a known parameter of the defibrillator.

Step 160 may include outputting to the output device additional heart condition information related to one or more time periods that differ from the certain time period. The one or more time periods may include a time period that follows the provision of the countershock.

The method may include a step of receiving from the defibrillator an indication that the certain heart condition information is related to the triggering of the provision of the countershock.

Method 100 may also include outputting an indication that the certain heart condition information is being stored.

The ECG monitor may include a processor, a frontend or other interface for receiving electrical signals from electrodes, a communication module, a memory unit, and the like. The ECG monitor may be a patch or any other unit.

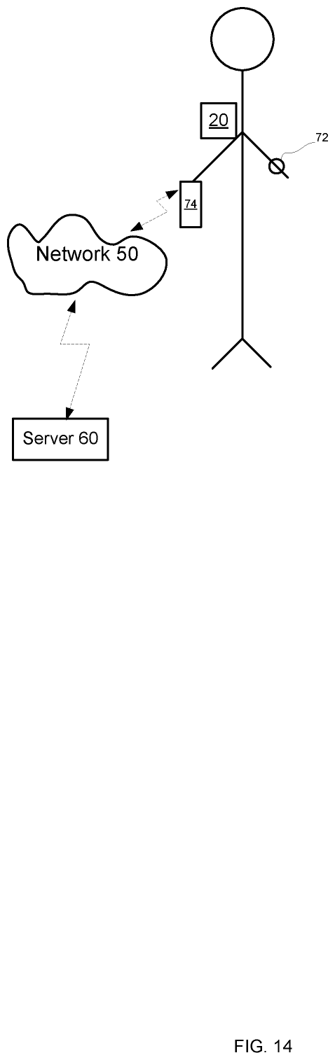

FIG. 14 illustrates a health monitor 72 located on the wrist of a person, a mobile phone 74 of the person, defibrillator 20, network 50, and server 60. The health monitor 62 may be a wearable monitor that may obtain heart condition information and may transmit it to the mobile phone 74, to the network, and the like. Heart condition information may be obtained by the health monitor 72 before a provision of a countershock, during the countershock and after the countershock—and may be used (for example by the server) to determine the condition of the heart in various point in time.

Any reference to the term “comprising” or “having” should be interpreted also as referring to “consisting” of “essentially consisting of”. For example—a method that comprises certain steps can include additional steps, can be limited to the certain steps or may include additional steps that do not materially affect the basic and novel characteristics of the method—respectively.

The invention may also be implemented in a computer program for running on a computer system, at least including code portions for performing steps of a method according to the invention when run on a programmable apparatus, such as a computer system or enabling a programmable apparatus to perform functions of a device or system according to the invention. The computer program may cause the storage system to allocate disk drives to disk drive groups.

A computer program is a list of instructions such as a particular application program and/or an operating system. The computer program may for instance include one or more of: a subroutine, a function, a procedure, an object method, an object implementation, an executable application, an applet, a servlet, a source code, an object code, a shared library/dynamic load library and/or other sequence of instructions designed for execution on a computer system.

The computer program may be stored internally on a non-transitory computer readable medium. All or some of the computer program may be provided on computer readable media permanently, removably or remotely coupled to an information processing system. The computer readable media may include, for example and without limitation, any number of the following: magnetic storage media including disk and tape storage media; optical storage media such as compact disk media (e.g., CD-ROM, CD-R, etc.) and digital video disk storage media; nonvolatile memory storage media including semiconductor-based memory units such as FLASH memory, EEPROM, EPROM, ROM; ferromagnetic digital memories; MRAM; volatile storage media including registers, buffers or caches, main memory, RAM, etc. A computer process typically includes an executing (running) program or portion of a program, current program values and state information, and the resources used by the operating system to manage the execution of the process. An operating system (OS) is the software that manages the sharing of the resources of a computer and provides programmers with an interface used to access those resources. An operating system processes system data and user input, and responds by allocating and managing tasks and internal system resources as a service to users and programs of the system. The computer system may for instance include at least one processing unit, associated memory and a number of input/output (I/O) devices. When executing the computer program, the computer system processes information according to the computer program and produces resultant output information via I/O devices.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

Also for example, the examples, or portions thereof, may implemented as soft or code representations of physical circuitry or of logical representations convertible into physical circuitry, such as in a hardware description language of any appropriate type.

Also, the invention is not limited to physical devices or units implemented in non-programmable hardware but can also be applied in programmable devices or units able to perform the desired device functions by operating in accordance with suitable program code, such as mainframes, minicomputers, servers, workstations, personal computers, notepads, personal digital assistants, electronic games, automotive and other embedded systems, cell phones and various other wireless devices, commonly denoted in this application as ‘computer systems’.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one current or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

We claim:

1. A method for heart monitoring, the method comprises:
monitoring a heart of a person to provide heart condition information;
storing the heart condition information;
sensing a provision of a countershock to the person, by a defibrillator, or receiving an indication from the defibrillator about the provision of the countershock; and
outputting to an output device that differs from the defibrillator, certain heart condition information related to a certain time period that precedes the provision of the countershock, wherein the certain heart condition information is related to a triggering of the provision of the countershock.

2. The method according to claim 1 wherein the monitoring of the heart of the person is executed by a defibrillator; and wherein the storing of the heart condition information comprises storing the heart condition information in a recording device.

3. The method according to claim 2 wherein the recording device is integrated with the defibrillator.

4. The method according to claim 2 wherein the recording device is mechanically connected to the defibrillator.

5. The method according to claim 2 wherein the recording device is detachably coupled to the defibrillator.

6. The method according to claim 2 wherein the recording device is mechanically connected to the defibrillator.

7. The method according to claim 1 comprising receiving from the defibrillator an indication that the certain heart condition information is related to the triggering of the provision of the countershock.

8. The method according to claim 1 wherein the defibrillator is an automated external defibrillator.

9. The method according to claim 1 wherein the certain time period precedes a timing of the provision of the countershock by a predetermined time difference.

10. The method according to claim 1 wherein the monitoring of the heart of the person comprises monitoring the heart of the person by a heart monitor that does not belong to the defibrillator, and wherein the heart condition information is a heart monitor gained heart condition information.

11. The method according to claim 10 comprising monitoring the heart rate of the person by the heart monitor, without receiving any information from the defibrillator.

12. The method according to claim 10 wherein the heart monitor is detachably connected to the person.

13. The method according to claim 10 comprising determining, by the defibrillator and based on defibrillator gained heart condition information, when to provide the countershock.

14. The method according to claim 13 wherein the heart monitor provided heart condition information is richer than the defibrillator gained heart condition information.

15. The method according to claim 10 comprising determining, by the heart monitor, and based on the heart monitor gained heart condition information, when to provide the countershock.

16. The method according to claim 15 comprising instructing the defibrillator, by the heart monitor, when to provide the countershock.

17. The method according to claim 10 comprising assigning timestamps to the heart condition information.

18. The method according to claim 1 comprising outputting to the output device additional heart condition information related to one or more time periods that differ from the certain time period.

19. The method according to claim 18 wherein the one or more time periods comprises a time period that follows the provision of the countershock.

20. The method according to claim 1 comprising outputting an indication that the certain heart condition information is being stored.

21. A non-transitory computer readable medium that stores instructions that once executed by a computerized system causes the computerized system to perform the steps of:
monitoring a heart of a person to provide heart condition information;
storing the heart condition information;
sensing a provision of a countershock to the person, by a defibrillator, or receiving an indication from the defibrillator about the provision of the countershock; and
outputting to an output device that differs from the defibrillator, certain heart condition information related to a certain time period that precedes the provision of the countershock, wherein the certain heart condition information is related to a triggering of the provision of the countershock.

22. The non-transitory computer readable medium according to claim 21 wherein the monitoring of the heart of the person is executed by a defibrillator; and wherein the storing of the heart condition information comprises storing the heart condition information in a recording device.

23. The non-transitory computer readable medium according to claim 21 that stores instructions for receiving from the defibrillator an indication that the certain heart condition information is related to the triggering of the provision of the countershock.

24. The non-transitory computer readable medium according to claim 21 wherein the defibrillator is an automated external defibrillator.

25. The non-transitory computer readable medium according to claim 21 wherein the certain time period precedes a timing of the provision of the countershock by a predetermined time difference.

26. The non-transitory computer readable medium according to claim 21 wherein the monitoring of the heart of the person comprises monitoring the heart of the person by a heart monitor that does not belong to the defibrillator, and wherein the heart condition information is a heart monitor gained heart condition information.

27. The non-transitory computer readable medium according to claim 26 that stores instructions for monitoring the heart rate of the person by the heart monitor, without receiving any information from the defibrillator.

28. The non-transitory computer readable medium according to claim 26 wherein the heart monitor is detachably connected to the person.

29. The non-transitory computer readable medium according to claim 26 that stores instructions for determining, by the defibrillator and based on defibrillator gained heart condition information, when to provide the countershock.

30. The non-transitory computer readable medium according to claim 29 wherein the heart monitor provided heart condition information is richer than the defibrillator gained heart condition information.

31. The non-transitory computer readable medium according to claim 26 that stores instructions for determining, by the heart monitor, and based on the heart monitor gained heart condition information, when to provide the countershock.

32. The non-transitory computer readable medium according to claim 31 that stores instructions for instructing the defibrillator, by the heart monitor, when to provide the countershock.

33. The non-transitory computer readable medium according to claim 26 that stores instructions for assigning timestamps to the heart condition information.

34. The non-transitory computer readable medium according to claim 21 that stores instructions for outputting to the output device additional heart condition information related to one or more time periods that differ from the certain time period.

35. The non-transitory computer readable medium according to claim 34 wherein the one or more time periods comprises a time period that follows the provision of the countershock.

36. The non-transitory computer readable medium according to claim 21 that stores instructions for outputting an indication that the certain heart condition information is being stored.

37. A system comprising a defibrillator and a recording device, wherein the defibrillator is configured to (a) monitor a heart of a person to provide heart condition information, (b) determine, based on the heart condition information, whether to generate a countershock, and (c) generate a countershock when determining to generate the countershock; wherein the recording device is configured to (a) receive from the defibrillator at least certain heart condition information that caused the defibrillator to determine to generate the countershock; (b) store the certain heart condition information; and (c) output the certain heart condition information.

38. The system according to claim 37 wherein the recording device is integrated with the defibrillator.

39. The system according to claim 37 wherein the recording device is mechanically connected to the defibrillator.

40. The system according to claim 37 wherein the recording device is detachably coupled to the defibrillator.

41. The system according to claim 37 wherein the recording device is mechanically connected to the defibrillator.

42. The system according to claim 37 wherein the defibrillator is configured to send to the recording device an indication that the certain heart condition information is related to the triggering of the provision of the countershock.

43. The system according to claim 37 wherein the defibrillator is an automated external defibrillator.

44. The system according to claim 37 wherein the certain time period precedes a timing of the provision of the countershock by a predetermined time difference.

45. The system according to claim 37 wherein the recording device is configured to output to the output device additional heart condition information related to one or more time periods that differ from the certain time period.

46. The system according to claim 45 wherein the one or more time periods comprises a time period that follows the provision of the countershock.

47. The system according to claim 37 wherein the recording device is configured to output an indication that the certain heart condition information is being stored.

48. A system comprising a defibrillator and a heart monitor that does not belong to the defibrillator;
wherein the heart monitor is configured to monitor a heart of a person to provide heart monitor gained heart condition information;
wherein the defibrillator is configured to monitor the heart of a person to provide defibrillator gained heart condition information;
wherein at least one device of the defibrillator and the heart monitor is configured to determine, based on at least one heart condition information out of the defibrillator gained heart condition information and the heart monitor gained heart condition information, whether to generate a countershock;
wherein the defibrillator is configured to generate a countershock when determining to generate the countershock; and
wherein the heart monitor is configured to output at least certain heart monitor gained heart condition information that caused the defibrillator to determine to generate the countershock.

49. The system according to claim 48 wherein the heart monitor is configured to determine, whether to generate a countershock; and to instruct the defibrillator to provide the countershock when determining to provide the countershock.

50. The system according to claim 48 wherein the defibrillator is configured to determine, whether to generate a countershock.

51. The system according to claim 48 wherein the heart monitor is configured to monitor the heart rate of the person without receiving any information from the defibrillator.

52. The system according to claim 48 wherein the heart monitor is detachably connected to the person.

53. The system according to claim 48 wherein the heart monitor provided heart condition information is richer than the defibrillator gained heart condition information.

54. The system according to claim 48 wherein the defibrillator is an automated external defibrillator.
